(19) United States
(12) Patent Application Publication
(10) Pub. No.: US 2004/0186300 A1
(43) Pub. Date: Sep. 23, 2004

(54) Process for the preparation of zafirlukast

(76) Inventors: Arie Gutman, Haifa (IL); Gennady Nisnevich, Haifa (IL); Victor Ponomarev, Haifa (IL); Maxim Sotrihin, Haifa (IL); Igor Zaltsman, Haifa (IL)

Correspondence Address: BROWDY AND NEIMARK, P.L.L.C., 624 NINTH STREET, NW, SUITE 300, WASHINGTON, DC, 20001-5303, US

(21) Appl. No.: 10/433,686

(22) PCT Filed: Nov. 30, 2001
(86) PCT No.: PCT/IB01/02268

(30) Foreign Application Priority Data
Dec. 5, 2000 (IL) 140096

(51) Int. Cl. C07D 209/04

(52) U.S. Cl. 548/452

(57) ABSTRACT

The present invention provides a novel process for the preparation of alkyl (1-alkylindol-3-ylmethyl)benzoate derivatives which process comprises the steps of: (a) reacting of an alkyl (halomethyl)benzoate with excess of an indole, said indole being unsubstituted at positions 1-, 2- and 3-, under conditions promoting alkylation at the 3-position of the indole to yield a mixture comprising alkyl (indol-3-ylmethyl)benzoate and unreacted starting indole, (b) treating the mixture obtained in step (a) with base to yield a mixture, comprising the salt of (indol-3-ylmethyl)benzoic acid and the unreacted indole, (c) recovering the unreacted indole from the mixture obtained in step (b), and recycling the indole as starting material to step (a), (d) isolating the salt of (indol-3-ylmethyl)benzoic acid and/or acidifying the salt to form (indol-3-ylmethyl)benzoic acid, (e) reacting the (indol-3-ylmethyl)benzoic acid or it's salt with alkylating agent in the presence of base to form the desired alkyl (1-alkylindol-3-ylmethyl)benzoate. The above process affords also the preparation of the anti-asthmatic leukotriene antagonist zafirlukast. In such case, methyl 3-methoxy-4-(1-methyl-5-nitroindol-3-ylmethyl)benzoate [a]is formed in step (e) of the process and this compound is subsequently converted into zafirlukast by known methods.

### FIELD OF THE INVENTION

The present invention relates to a novel process for the preparation of (1-alkylindol-3-ylmethyl)benzoic acid derivatives, in particular zafirlukast and precursors thereof, to novel intermediates used in this process and to the preparation thereof.

### LIST OF REFERENCES

Matassa, V. G. et al., J. Med. Chem., v. 33, 1781(1990);

U.S. Pat. No. 4,859,692;

U.S. Pat. No. 5,993,859;

The Merck Index, 12th Edition, 10241.

### BACKGROUND OF THE INVENTION

Zafirlukast, cyclopentyl 3-[2-methoxy-4-[(o-tolylsulfonyl)carbamoyl]-benzyl]-1-methylindole-5-carbamate, having the formula:

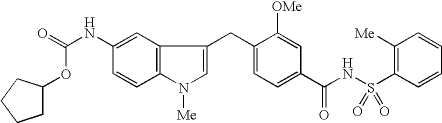

is a first anti-asthmatic leukotriene antagonist (Matassa, V. G. et al., J. Med. Chem., v. 33, 1781 (1990); U.S. Pat. No. 4,859,692 and The Merck Index, 12th Edition, 10241).

Methods for the preparation of Zafirlukast are described in J. Med. Chem., v. 33, 1781 (1990), U.S. Pat. No. 4,859,692 and U.S. Pat. No. 5,993,859 starting from methyl 3-methoxy-4-(1-methyl-5-nitroindol-3-ylmethyl)benzoate [1a]

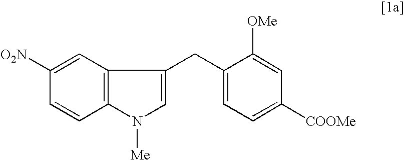

Alkyl (1-alkylindol-3-ylmethyl)benzoates of formula [1b] are useful as chemical intermediates in the pharmaceutical industry.

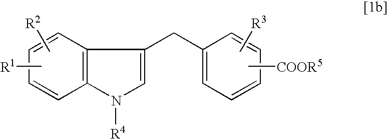

These compounds may be obtained by a process described in J. Med. Chem., v. 33, 1781 (1990) and U.S. Pat. No. 4,859,692. This process comprises the steps of:

(a) reacting an alkyl (halomethyl)benzoate of formula [2] with an equivalent amount of an indole of formula [3]

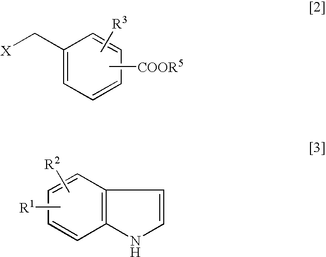

in the presence of an equivalent quantity of silver(I) oxide,

(b) isolating the alkyl (indol-3-ylmethyl)benzoates of formula [4] from the reaction mixture obtained in step (a) above,

(c) reacting the compound [4] with an alkylating agent of formula [6],

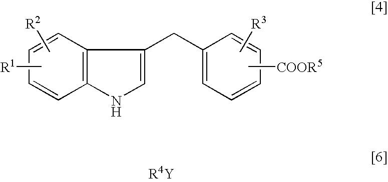

The above process has serious disadvantages in the isolation of the product [4] in step (b) which is due to the fact that alkylation of indole, that is unsubstituted at positions 1-, 2- and 3-, at the 3-position, is accompanied by the undesired process of polyalkylation, to form polysubstituted indoles of formula [7]and/or formula [8]:

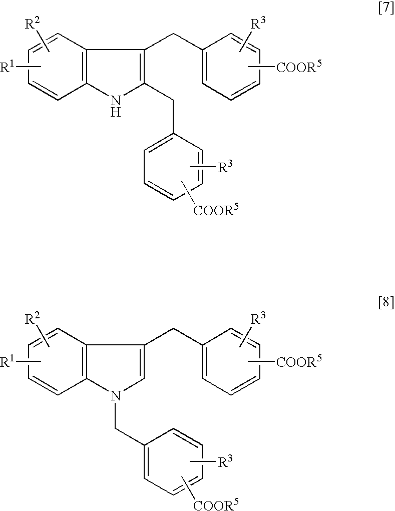

while at the same time some quantity of the starting unreacted indole remains in the reaction mixture. Most common methods for the separation of alkyl (indol-3-ylmethyl)benzoate of formula [4] from by-products of polyalkylation and starting unreacted indole, which are all covalent compounds with similar physical properties, include column chromatography that is an unpractical method for industrial scale applications.

It should be noted, that silver(I) ion, while promoting the desired alkylation of the indoles [3] at the 3-position, also catalyzes the by-processes of isomerization and disproportionation of the alkyl (indol-3-ylmethyl)benzoates

### SUMMARY OF INVENTION

It is an object of the present invention to provide a novel process for the preparation of (1-alkylindol-3-ylmethyl)benzoic acid-derivatives, in particular zafirlukast, which is free of the above-mentioned disadvantages.

The above object is achieved in accordance with the present invention which, in one aspect thereof, provides a process for preparing an alkyl (1-alkylindol-3-ylmethyl)benzoate, which process comprises the steps of:

(a) reacting an alkyl (halomethyl)benzoate with excess of an indole, said indole being unsubstituted at positions 1-, 2- and 3-, under conditions promoting alkylation at the 3-position of the indole to yield a mixture comprising alkyl (indol-3-ylmethyl)benzoate and unreacted starting indole,

(b) treating the mixture obtained in step (a) with base to yield a mixture, comprising a salt of (indol-3-ylmethyl)benzoic acid and the unreacted indole,

(c) recovering the unreacted indole from the mixture obtained in step (b), and recycling the indole as starting material to step (a),

(d) isolating the salt of (indol-3-ylmethyl)benzoic acid and optionally acidifying the salt to form (indol-3-ylmethyl)benzoic acid,

(e) reacting the (indol-3-ylmethyl)benzoic acid or it's salt with an alkylating agent in the presence of base to form the desired alkyl (1-alkylindol-3-ylmethyl)benzoate.

Preferably, when methyl 3-methoxy-4-(1-methyl-5-nitroindol-3-ylmethyl)benzoate is formed in step (e) above, it is then converted into zafirlukast.

The proposed process excludes using unpractical on industrial scale column chromatography for isolation of the desired product and its intermediates.

In accordance with another aspect of this invention, the present invention provides a novel compound, namely the sodium salt of 3-methoxy-4-(5-nitroindol-3-ylmethyl)benzoic acid. This novel compound is obtained as an intermediate in the process of the present invention. It is a stable, solid compound, obtainable in high yield, which can be easily purified by crystallization and stored for long periods of time.

### DETAILED DESCRIPTION OF THE INVENTION

The invention provides a process for the preparation of alkyl (1-alkylindol-3-ylmethyl)benzoate which comprises:

(a) reacting an alkyl (halomethyl)benzoate with excess of an indole, said indole being unsubstituted at positions 1-, 2- and 3-, under conditions promoting alkylation at the 3-position of the indole to yield a mixture comprising alkyl (indol-3-ylmethyl)benzoate and unreacted starting indole,

(b) treating the mixture obtained in step (a) with base to yield a mixture, comprising a salt of (indol-3-ylmethyl)benzoic acid and the unreacted indole,

(c) recovering the ureacted indole from the mixture, obtained in step (b), and recycling the indole as starting material to step (a),

(d) isolating the salt of (indol-3-ylmethyl)benzoic acid and/or acidifying the salt to afford (indol-3-ylmethyl)benzoic acid,

(e) reacting the (indol-3-ylmethyl)benzoic acid or it's salt with an alkylating agent in the presence of base to afford the desired alkyl (1-alkylindol-3-ylmethyl)benzoate.

The reaction in step (a) may be carried out without addition of any other reagents or in the presence of a Lewis acid and, optionally, a base. The Lewis acid may be silver(I) oxide, silver(I) salt or zinc halide. It should be noted, that silver(I) ion, while promoting the desired alkylation of the indoles at the 3-position, also catalyzes the by-processes of isomerization and disproportionation of the alkyl (indol-3-ylmethyl)benzoates. Preferably, the reaction in step (a) is carried out in the presence of zinc halide and base. Alkyl (indol-3-ylmethyl)benzoates demonstrate stability in the presence of zinc halide and base in contrast to isomerization and disproportionation processes which occur to a significant degree in the presence of silver(I) ion. Preferably, the zinc halide is zinc iodide, zinc bromide or zinc chloride or mixtures thereof.

Preferably, the base is an organic base, for example a tertiary amine or heterocyclic amine. The tertiary amine is preferably a sterically hindered tertiary amine.

The reaction in step (a) is preferably conducted in an aprotic solvent, such as for example ether, ester, aromatic hydrocarbon, halogenated solvent or mixtures thereof.

Although any indoles, being unsubstituted at positions 1-, 2- and 3-, can be used in the process of the invention, preferably compounds of formula [3]

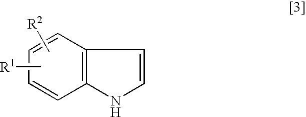

are used, wherein R¹ and R² are independently selected from a group consisting of hydrogen, alkyl, aryl, aralkyl, halogen, alkoxy, aryloxy, nitro, and disubstituted amino group. Preferably, R¹ is nitro and R² is hydrogen or halogen. More preferably, the indole is 5-nitroindole.

Although any alkyl (halomethyl)benzoate can be used in the process of the invention, preferably compounds of formula [2]

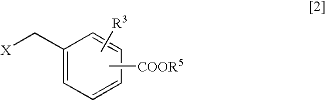

are used, wherein R³ is selected from hydrogen, alkyl, halogen, alkoxy and trifluoromethyl; R⁵ is alkyl; and X is halogen, for example chloro, bromo or iodo, preferably bromo or iodo. R³ is preferably an alkoxy group. More preferably, R³ is methoxy. Most preferably, the alkyl (halomethyl)benzoate is an alkyl 4-halomethyl-3-methoxybenzoate. Preferably, at least 1.1 mole of the indole, being unsubstituted at positions 1-, 2- and 3-, may be used per mole of the alkyl (halomethyl)benzoate. More preferably, the mole ratio of the indole to the alkyl (halomethyl)benzoate should be in the range of 1.3 to 1.7/1.

Although any alkylating agents can be used in the process of the invention, preferably alkylating agents of formula [6]

R⁴Y [6]

are used, wherein R⁴ is selected from alkyl, aralkyl, haloalkyl, cyanoalkyl, (alkoxycarbonyl)alkyl, (arylcarbonyl)alkyl, alkanoylalkyl, [(dialkylamino)carbonyl]alkyl, [(3-8C)cycloalkyl]alkyl and alkoxyalkyl; and Y is a displaceable group; Preferably, R⁴ is alkyl group. Most preferably, the alkylating agent is a methylating agent.

The invention provides an efficient method for the preparation of numerous known and new alkyl (1-alkylindol-3-ylmethyl)benzoates.

Particularly, alkyl (1-alkylindol-3-ylmethyl)benzoates of formula [1]

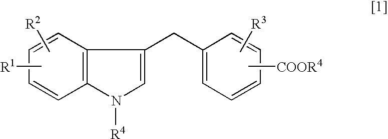

may be obtained by

(a) reacting an alkyl (halomethyl)benzoate of formula [2]

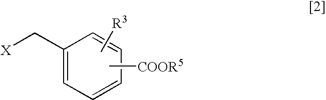

with excess of an indole of formula [3]

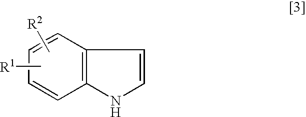

under conditions promoting alkylation at the 3-position of the indole, to yield a mixture, comprising the alkyl (indol-3-ylmethyl)benzoate of formula [4]

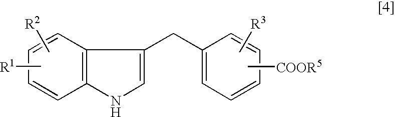

and unreacted starting indole [3],

(b) treating the mixture, obtained in step (a), with base to yield a mixture, comprising the salt of the acid of formula [5]

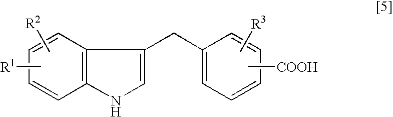

and the unreacted indole [3],

(c) recovering the unreacted indole [3] from the mixture, obtained in step (b), and recycling it as starting material to step (a),

(d) isolating the salt of (indol-3-ylmethyl)benzoic acid [5] and/or acidifying the salt to form the (indol-3-ylmethyl)benzoic acid [5],

(e) reacting the (indol-3-ylmethyl)benzoic acid [5] or it's salt with an alkylating agent of formula [6]

R⁴Y [6]

wherein R¹, R², R³, R⁴, R⁵, X and Y are as previously defined,

in the presence of base to form the desired alkyl (1-alkylindol-3-ylmethyl)benzoates of formula [1].

The described process for the production of alkyl (1-alkylindol-3-ylmethyl)benzoates [1] may be summarized by the following Scheme 1:

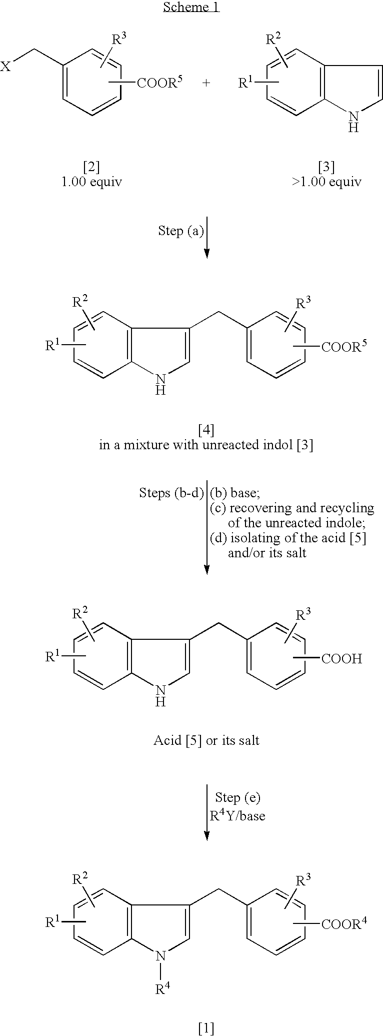

The above process affords also the preparation of zafirlukast. In such case, the compound methyl 3-methoxy-4-(1-methyl-5-nitroindol-3-ylmethyl)benzoate [a]is formed in step (e) of the process and this compound is subsequently converted into zafirlukast by known methods.

The preparation of zafirlukast is showed in the following Scheme 2:

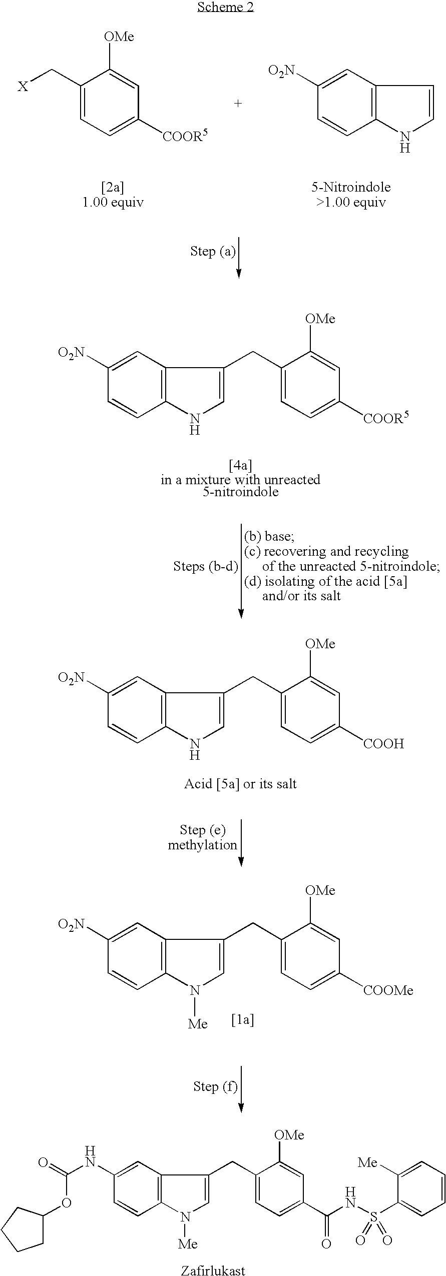

In comparison, the known process for the preparation of zafirlukast described in J. Med. Chem., v. 33, 1781 (1990) and U.S. Pat. No. 4,859,692 involves separation steps, e.g. column chromatography, that are not practical for industrial scale applications. The known process is summarized in Scheme 3:

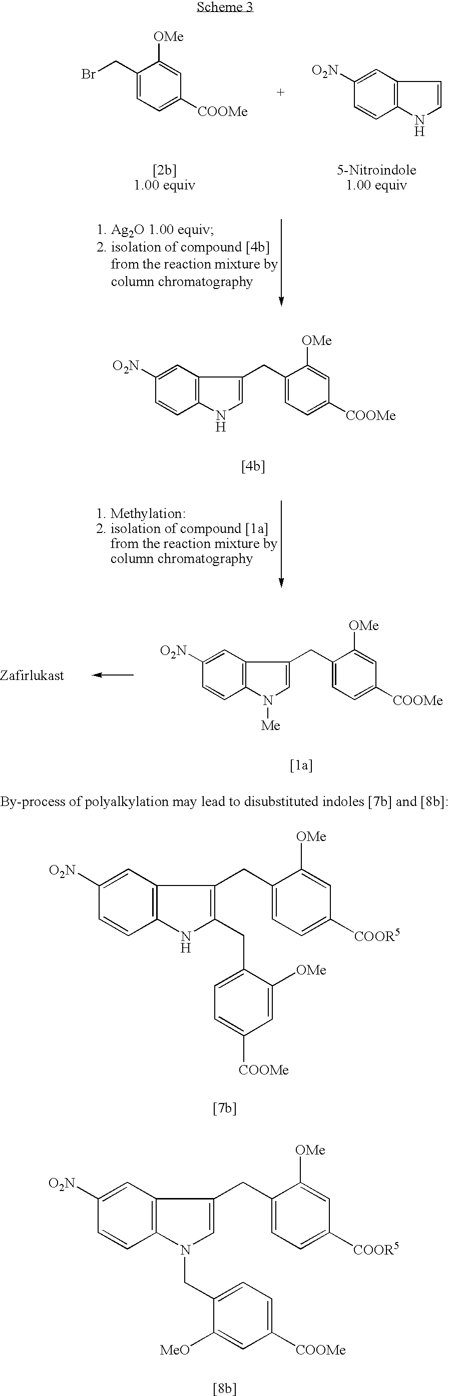

This invention will be better understood from the Examples that follow. However, the examples illustrate, but do not limit, the invention. Those skilled in the art will readily appreciate that the specific methods and results discussed are merely illustrative of the invention as described more fully in the claims that follow thereafter.

### EXAMPLE 1

Preparation of methyl 4-(bromomethyl)-3-methoxybenzoate [2b]

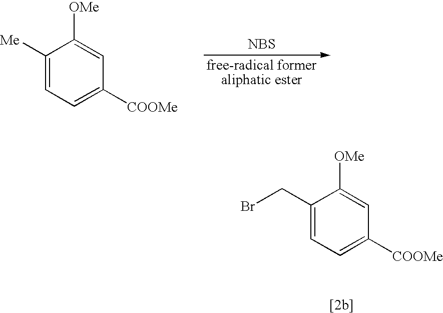

A mixture of methyl 3-methoxy-4-methylbenzoate (1000 g, 5.55 mol), N-bromosuccinimide (NBS) (1185 g, 6.66 mol), benzoyl peroxide (20.0 g, 0.08 mol) and ethyl acetate (5.0 L) was stirred under reflux conditions for 5 hours. The mixture was cooled to the room temperature, washed with water, dried over sodium sulfate, filtered and concentrated under reduced pressure to the volume of 2 L. Hexane (4 L) was added to the stirred solution under reflux conditions. The mixture was allowed to stay overnight at −5-−10° C. The precipitated crystals were filtered off, washed on filter with cooled hexane and dried under reduced pressure to yield from 1107 to 1136 g (77-79%) of methyl 4-(bromomethyl)-3-methoxybenzoate [2b] as off white crystals with 91-94.5% purity by GC.

### EXAMPLE 2

Preparation of sodium 3-methoxy-4-(5-nitroindol-3-ylmethyl)benzoate in a Solid Physical Form

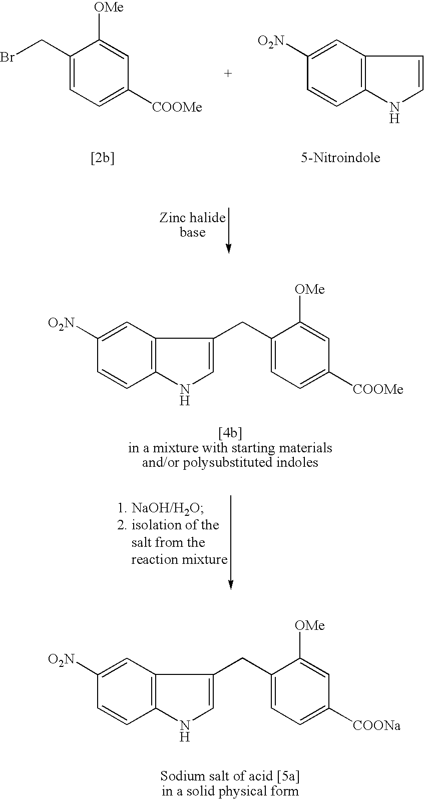

A mixture of 5-nitroindole (1038.9 g, 6.41 mol), zinc bromide (576.5 g, 2.56 mol), N,N-diisopropylethylamine (828.1 g, 6.41 mol) and 1,4-dioxane (7.0 L) was stirred for 20 min at room temperature. Methyl 3-methoxy-4-(bromomethyl)benzoate [2b] (1000.0 g, 3.86 mol) was added in one portion to the stirred mixture. The mixture was stirred for 40 hours at 20-25° C. and evaporated under reduced pressure at 40-50° C. The solution of the residue in dichloromethane (6 L) was washed consistently with 5% hydrochloric acid (2×3 L) (Note 1) and water (3 L), dried over sodium sulfate, passed through a short silica gel column and evaporated under reduced pressure. A mixture of the residue, sodium hydroxide (308.0 g), methanol (5.0 L) and water (2.5 L) was stirred for 1-2 hours at 70° C. Methanol was evaporated from the mixture under reduced pressure. The resulting suspension was stirred for 1 hour at 65° C. and filtered at the same temperature (Note 2). The filtrate was kept for 30 hours at 20-25° C. The precipitated solid was filtered off, washed on filter with cold water and dried under reduced pressure at 70-80° C. to give 807.0 g (60% yield) of crude sodium 3-methoxy-4-(5-nitroindol-3-ylmethyl)benzoate as yellow solid. Re-crystallization of the crude material from boiling water gives an analytical sample of the sodium 3-methoxy-4-(5-nitroindol-3-ylmethyl)benzoate with mp 330° C. (dec.) and 99.0% purity by HPLC. ¹H NMR (DMSO-d₆, δ ppm): 3.85 (s, 3H), 4.03 (s, 2H), 7.02 (d, 1H, J 8.2 Hz), 7.32-7.40 (m, 2H), 7.47-7.55 (m, 2H), 7.93 (dd, J 8.94 and 2.2 Hz, 1H), 8.48 (d, J 2.2 Hz, 1H), 12.04 (s, 1H). Infra red spectrum confirmed proposed structure.

Notes:

1. The acidic solution was basified with sodium hydroxide to pH 11 and extracted with dichloromethane (3×1 L). The organic layers were separated, dried over sodium sulfate, filtered and fractionally rectified to give 490 g (59.2% recovery) of N,N-diisopropylethylamine.

2. The filtered off crystalline 5-nitroindole was washed on filter with warm (60° C.) water (500 mL). A solution of the 5-nitroindole in chloroform (3.0 L) was washed with water (2×2 L), dried over sodium sulfate, filtered and concentrated up to volume of 1 L. Hexane (4 L) was added to the stirred solution and the mixture was kept for 2 hours at room temperature. The precipitated crystals were filtered off, washed on filter with hexane and dried under reduced pressure to give 405 g (98% recovery) of 5-nitroindole as yellow crystals with 99% purity by HPLC.

### EXAMPLE 3

Preparation of 3-methoxy-4-(5-nitroindol-3-ylmethyl)benzoic acid [5a]

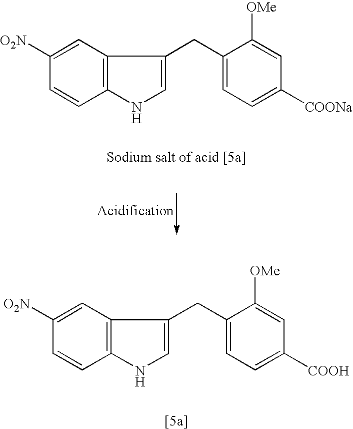

The crude sodium 3-methoxy-4-(5-nitroindol-3-ylmethyl)benzoate (805.0 g) from the previous step was dissolved in a mixture of water (5.0 L) and methanol (200 ml) at 60-70° C. and extracted with chloroform (3×1.0 L). The water layer was separated and acidified with 32% hydrochloric acid to pH ˜1. The precipitated solid was filtered off, washed on filter with cold water and dried under reduced pressure to give 710.0 g. (93.5% yield) of 3-methoxy-4-(5-nitro-3-indolylmethyl)benzoic acid [5a] as yellow solid with 87% purity by HPLC. Analogously, from the 99.0% pure sodium 3-methoxy-4-(5-nitroindol-3-ylmethyl)benzoate analytical sample of crystalline acid [5a] with mp 258-259° C. and 99.3% purity by HPLC was obtained. ¹H NMR (DMSO-d₆, δ ppm): 3.90 (s, 3H), 4.10 (s, 2H), 7.22 (d, J 7.7 Hz, 1H), 7.40-7.55 (m, 4H), 7.95 (dd, J 9.0 and 2.3 Hz, 1H), 8.47 (d, J 2.32 Hz, 1H), 11.63 (s, 1), 12.88 (s, 1H). Infra red spectrum confirmed proposed structure.

### EXAMPLE 4

Preparation of methyl 3-methoxy-4-(1-methyl-5-nitroindol-3-ylmethyl)benzoate [a]

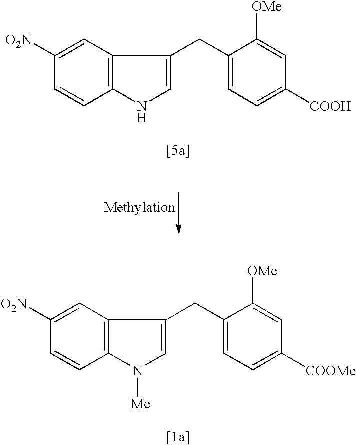

A mixture of 3-methoxy-4-(5-nitroindol-3-ylmethyl)benzoic acid [5a] (200.0 g, 0.61 mol), dimethyl sulfate (232.1 g, 1.84 mol), potassium carbonate (279.1 g, 2.02 mol) and 2-butanone (1.6 L) was stirred under reflux conditions for 5 hours and evaporated under reduced pressure. The residue was dissolved in a mixture of dichloromethane (2 L) and water (2 L). The organic layer was separated, washed with water, dried over sodium sulfate, passed through a short silica gel column and concentrated under reduced pressure to the volume of 0.5 L. Hexane (2 L) was added to the stirred mixture under reflux conditions. The mixture was allowed to stay overnight at 20-25° C. The precipitated crystals was filtered off, washed on filter with hexane and dried under reduced pressure to give 208.0 g (96.2% yield) of methyl 3-methoxy-4-(1-methyl-5-nitroindol-3-ylmethyl)benzoate [a]with 96% purity by HPLC. The crude compound was re-crystallized from a mixture of dichloromethane and hexane to give purified compound [a]with mp 145-153° C. NMR ¹H (CDCl₃, δ ppm): 3.75 (s, 3H), 3.87 (s, 3H), 3.93 (s, 3H), 4.10 (s, 2H), 6.90 (s, 1H), 7.14 (d, J 8.2 Hz, 1H), 7.23 (d, J 9.0 Hz, 1H), 7.50-7.58 (m, 2H), 8.06 (dd, J 9.0 and 2.34 Hz, 1H), 8.54 (d, J 2.34 Hz, 1H). Infra red spectrum confirmed proposed structure.

### EXAMPLE 5

Preparation of methyl 3-methoxy-4-(5-amino-1-methylindol-3-ylmethyl)benzoate [11]

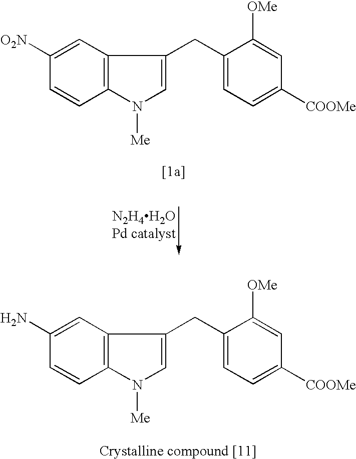

Hydrazine monohydrate, 80% aq. solution (48.4 g, 0.77 mol) was added dropwise to a stirred mixture of methyl 3-methoxy-4-(1-methyl-5-nitroindol-3-ylmethyl)benzoate [a] (137.0 g, 0.38 mol), palladium, 10 wt. % on carbon powder (13.7 g) and absolute ethanol (1.0 L) during 2 hours at 65-70° C. The mixture was stirred under reflux conditions for 5 hours and filtered at 50° C. through filter agent Celite® to remove the catalyst. The catalyst was washed on the filter with dichloromethane (1 L). The combined filtrates were evaporated under reduced pressure to give 115.5 g (91% yield) of 3-methoxy-4-(5-amino-1-methylindol-3-ylmethyl)benzoate [11] with 97.5% purity by HPLC. Further crystallization of the compound from a mixture of dichloromethane and hexane give crystalline compound [11] with mp 129-130° C. ¹H NMR (CDCl₃, 6 ppm): 3.66 (s, 3H), 3.88 (s, 3H), 3.92 (s, 3H), 4.02 (s, 2H), 6.64-6.78 (m, 3H), 7.06-7.14 (m, 2H), 7.48-7.53 (m, 2H). Infra red spectrum confirmed proposed structure.

### EXAMPLE 6

Preparation of methyl 4-(5-cyclopentyloxycarbonylamino-1-methylindol-3-ylmethyl)-3-methoxybenzoate [12]

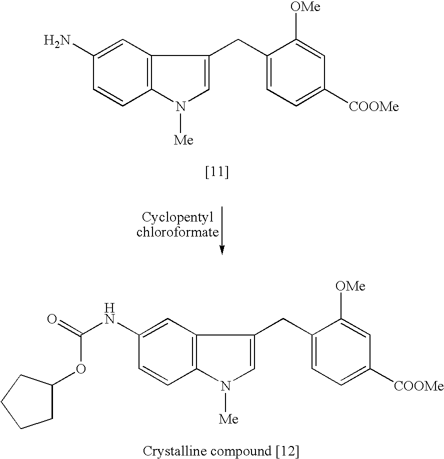

A solution of cyclopentyl chloroformate (55.8 g, 0.31 mol) in dichloromethane (50 mL) was added dropwise under argon atmosphere to the stirred mixture of methyl 3-methoxy-4-(5-amino-1-methylindol-3-ylmethyl)benzoate [11] (100.0 g, 0.31 mol), N-methylmorpholine (77.9 g, 0.77 mol) and dichloromethane (500 mL) at 10° C. The mixture was stirred for 2 hours at 20-25° C., washed with 5% hydrochloric acid (3×500 mL) and water (2×300 mL). Organic layer was separated, dried over sodium sulfate, passed through a short silica gel column and concentrate under reduced pressure to the volume of 200 mL. Hexane (1 L) was added to the stirred mixture. The formed precipitate was filtered off, washed on filter with hexane and dried under reduced pressure to give 127.0 g (95% yield) of methyl 4-(5-cyclopentyloxycarbonylamino-1-methylindol-3-ylmethyl)-3-methoxybenzoate [12] with 97.8% purity by HPLC. The compound was re-crystallized from a mixture of dichloromethane and hexane to give the crystalline compound [12] with mp 134-135° C. ¹H NMR (CDCl₃, δ, ppm): 1.58-1.88 (m, 8H), 3.68 (s, 3H), 3.87 (s, 3H), 3.91 (s, 3H), 4.05 (s, 2H), 5.15-5.21 (m, 1H), 6.49 (s, 1H), 6.73 (s, 1H), 7.09-7.23 (m, 3H), 7.48-7.52 (m, 3H). Infra red spectrum confirmed proposed structure.

### EXAMPLE 7

Preparation of sodium 4-(5-cyclopentyloxycarbonylamino-1-methylindol-3-ylmethyl)-3-methoxybenzoate in a Solid Physical Form

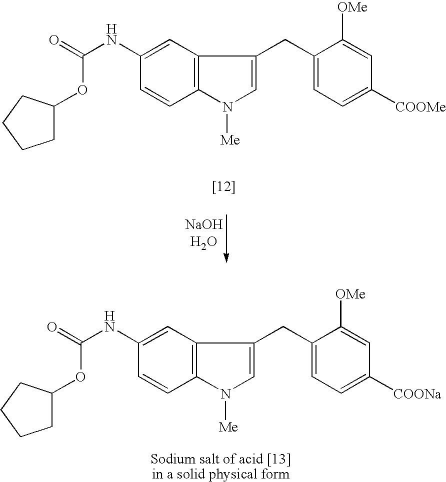

A mixture of methyl 4-(5-cyclopentyloxycarbonylamino-1-methylindol-3-ylmethyl)-3-methoxybenzoate [12] (116.0 g, 0.27 mol), sodium hydroxide (31.9 g, 0.80 mol), 1,4-dioxane (300 mL), methanol (50 mL) and water (100 mL) was stirred for 12 hours at 20-25° C. 1,4-Dioxane and methanol were removed from the mixture under reduced pressure. Cold water (400 mL) was added to the mixture. The obtained suspension was stirred at 0-5° C. for 20 min. The formed precipitate was filtered off, thoroughly washed on filter with cold water and dried under reduced pressure over the phosphorous pentoxide to give 114.0 g (95% yield) of sodium 4-(5-cyclopentyloxycarbonylamino-1-methylindol-3-ylmethyl)-3-methoxybenzoate as off white crystalline compound with mp 187-188° C. and 99.5% purity by HPLC. ¹H NMR (DMSO-d₆, δ, ppm): 1.55-1.82 (m, 8H), 3.66 (s, 3H), 3.84 (s, 3H), 3.88 (s, 2H), 4.98-5.12 (m, 1H), 6.92 (d, J 7.70 Hz, 1H), 6.95 (s, 1H), 7.14 (d, J 8.70 Hz, 1H), 7.24 (d, J 8.70 Hz, 1H), 7.36 (d, J 7.70 Hz, 1H), 7.53 (s, 1H), 7.64 (s, 1H), 9.21 (s, 1H). Infra red spectrum confirmed proposed structure.

### EXAMPLE 8

Preparation of 4-(5-cyclopentyloxycarbonylamino-1-methylindol-3-ylmethyl)-3-methoxybenzoic acid [13]

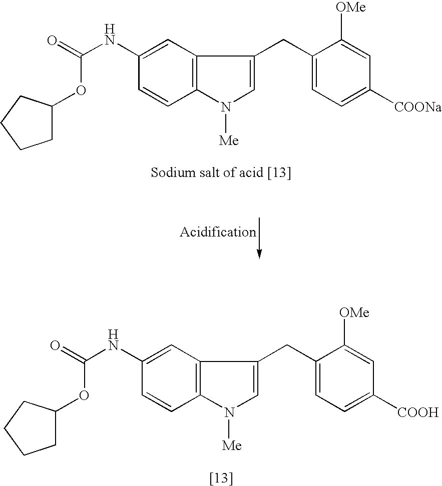

A solution of sodium 4-(5-cyclopentyloxycarbonylamino-1-methylindol-3-ylmethyl)-3-methoxybenzoate in boiling water (1.2 mL) was acidified with 32% hydrochloric acid to pH ˜1-2. The mixture was stirred for 0.5 hour at 0-5° C. The precipitated white fine crystals were filtered off, thoroughly washed on filter with cold water and dried under reduced pressure over the phosphorous pentoxide to give 105.5 g (94% yield) of 4-(5-cyclopentyloxycarbonylamino-1-methylindol-3-ylmethyl)-3-methoxybenzoic acid [13] with mp 204-205° C. and 99.5% purity by HPLC. ¹H NMR (DMSO-d₆, δ, ppm): 1.55-1.83 (m, 8H), 3.68 (s, 3H), 3.90 (s, 3H), 3.95 (s, 2H), 4.98-5.10 (m, 1H), 7.02 (s, 1H), 7.10 (d, J 7.86 Hz, 1H), 7.14-7.18 (m, 1H), 7.26 (d, J 8.6 Hz, 1H), 7.43 (d, J 7.9 Hz, 1H), 7.48 (s, 1H), 7.60 (s, 1H), 9.21 (s, 1H), 12.70-12.91 (m, 1H). Infra red spectrum confirmed proposed structure.

### EXAMPLE 9

Preparation of Zafirlukast

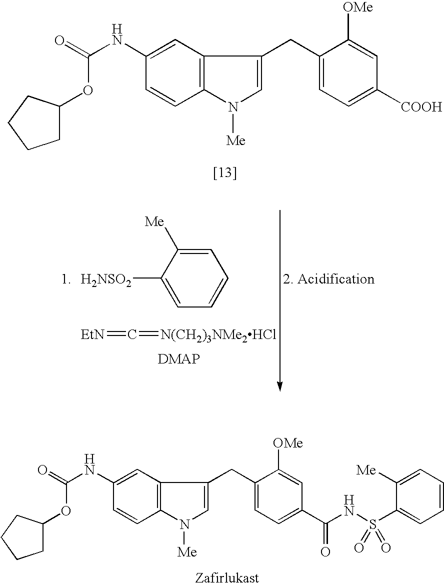

A mixture of 4-(5-cyclopentyloxycarbonylamino-1-methyl-3-indolylmethyl)-3-methoxybenzoic acid [13] (700 g, 1.66 mol), o-toluenesulfonamide (297.9 g, 1.74 mol), 4-(dimethylamino)pyridine (DMAP) (212.5 g, 1.74 mol), 1-[(3-dimethylamino)propyl]-ethylcarbodiimide hydrochloride (333.5 g, 1.74 mol) and dichloromethane (3.5 L) was stirred for 24 hours at 20-25° C. under inert atmosphere. 5% Hydrochloric acid (2 L) was added dropwise to the stirred mixture at 5-10° C. The obtained mixture was stirred for 20 min. The organic layer was separated, washed with water (1 L), quickly dried over sodium sulfate, passed through short silica gel column and concentrated under reduced pressure to the volume of 2 L. Absolute ethanol was added to the stirred solution under reflux conditions. Dichloromethane was distilled of from the stirred mixture through 10 cm Vigreux column equipped with a distillation head until the temperature of vapor in the head of the column was reached 78° C. The resulting mixture was kept overnight at the room temperature. The precipitated crystals were filtered off, washed on filter with cold absolute ethanol and re-crystallized from the mixture of dichloromethane and ethanol according to above mentioned procedure, to give after drying under reduced pressure 775.2 g (81.3% yield) of zafirlukast ethanolate as white powder with mp 132-133° C. (dec.) and 99.8% purity by HPLC. ¹H NMR (CDCl₃, δ, ppm): 1.22 (t, J 7.05 Hz, 3H), 1.45-1.87 (m, 8H), 2.66 (s, 3H), 3.67 (s, 3H), 3.73 (q, J 7.05 Hz, 4H), 3.79 (s, 3H), 3.98 (s, 2H), 5.08-5.23 (m, 1H), 6.58 (s, 1H), 6.73 (s, 1H), 7.01-7.51 (m, 9H), 8.23 (d, J 7.52 Hz, 1H), 9.67 (s, 1H).

1. A process for the preparation of an alkyl (1-alkylindol-3-ylmethyl)benzoate, such process comprising:
(a) reacting of an alkyl (halomethyl)benzoate with excess of an indole, said indole being unsubstituted at positions 1-, 2- and 3-, in the presence of zinc halide under conditions promoting alkylation at the 3-position of the indole to yield a mixture comprising alkyl (indol-3-ylmethyl)benzoate and unreacted starting indole,
(b) treating the mixture obtained in step (a) with sodium hydroxide to yield a mixture comprising the monosodium salt of (indol-3-ylmethyl)benzoic acid and the unreacted indole,
(c) recovering the unreacted indole from the mixture obtained in step (b), and recycling the indole as starting material to step (a),
(d) isolating the monosodium salt of (indol-3-ylmethyl)benzoic acid in substantially pure solid form and/or acidifying the salt to form (indol-3-ylmethyl)benzoic acid,
(e) reacting the (indol-3-ylmethyl)benzoic acid or it's salt with an alkylating agent in the presence of base to form the desired alkyl (1-alkylindol-3-ylmethyl)benzoate.

2. The process of claim 1 wherein in step (a) the reaction is carried out in the presence of a base.

3. The process of claim 1 wherein the zinc halide is selected from zinc iodide, zinc bromide, zinc chloride and mixtures thereof.

4. The process of claim 2 wherein the base is an organic base.

5. The process of claim 4 wherein the organic base is tertiary amine or heterocyclic amine.

6. The process of claim 5 wherein the tertiary amine is a sterically hindered tertiary amine.

7. The process of claim 1, wherein the reaction in step (a) is conducted in an aprotic solvent.

8. The process of claim 7, wherein said aprotic solvent is selected from ether, ester, aromatic hydrocarbon, halogenated solvent or mixtures thereof.

9. The process of claim 1 wherein the indole unsubstituted at positions 1-, 2- and 3- is a compound of formula [3]: 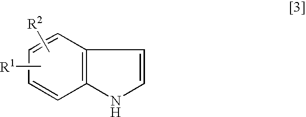
wherein R¹ and R² are independently selected from a group consisting of hydrogen, alkyl, aryl, aralkyl, halogen, alkoxy, aryloxy, nitro and disubstituted amino group.

10. The process of claim 9 wherein R¹ is nitro and R² is hydrogen or halogen.

11. The process of claim 1 wherein the indole unsubstituted at positions 1-, 2- and 3- is 5-nitroindole.

12. The process of claim 1 wherein the alkyl (halomethyl)benzoate is a compound of formula [2]: 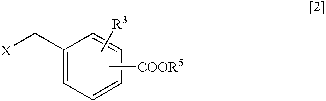
wherein R³ is selected from hydrogen, alkyl, halogen, alkoxy and trifluoromethyl; R⁵ is alkyl; and X is halogen.

13. The process of claim 12 wherein R³ is alkoxy group.

14. The process of claim 12 wherein R³ is methoxy group.

15. The process of claim 1 wherein the alkyl (halomethyl)benzoate is an alkyl 4-halomethyl-3-methoxybenzoate.

16. The process of claim 1 wherein the alkylating agent is a compound of formula [6]:
R⁴Y [6]
wherein R⁴ is selected from alkyl, aralkyl, haloalkyl, cyanoalkyl, (alkoxycarbonyl)alkyl, (arylcarbonyl)alkyl, alkanoylalkyl, [(dialkylamino)carbonyl]alkyl, [(3-8C)cycloalkyl]alkyl and alkoxyalkyl; and Y is a displaceable group.

17. The process of claim 16 wherein R⁴ is alkyl group.

18. The process of claim 1 wherein the alkylating agent is a methylating agent.

19. A process as claimed in claim 1 wherein the alkyl (1-alkylindol-3-ylmethyl)benzoate is a compound of formula [1] 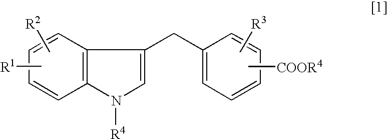
the alkyl (halomethyl)benzoate is a compound of formula [2] 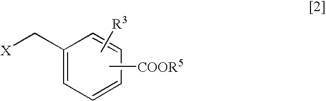
the indole unsubstituted at positions 1-, 2- and 3- is a compound of formula [3] 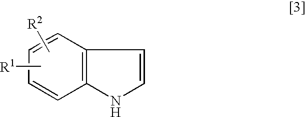
the alkyl (indol-3-ylmethyl)benzoate is a compound of formula [4]: 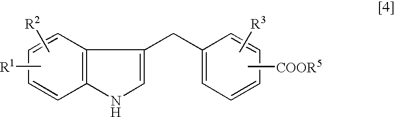
the (indol-3-ylmethyl)benzoic acid is a compound of formula [5] 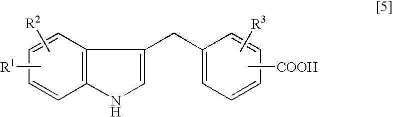
the alkylating agent is a compound of formula [6]
R⁴Y [6]
wherein R¹ and R² are independently selected from a group consisting of hydrogen, alkyl, aryl, aralkyl, halogen, alkoxy, aryloxy, nitro and disubstituted amino group; R³ is selected from hydrogen, alkyl, halogen, alkoxy and trifluoromethyl; R⁴ is selected from alkyl, aralkyl, haloalkyl, cyanoalkyl, (alkoxycarbonyl)alkyl, (arylcarbonyl)alkyl, alkanoylalkyl, [(dialkylamino)carbonyl]alkyl, [(3-8C)cycloalkyl]alkyl and alkoxyalkyl; R⁵ is alkyl; X is halogen and Y is a displaceable group.

20. The use of substantially pure solid monosodium salt of (indol-3-ylmethyl)benzoic acid as defined in claim 19, of formula [5a] 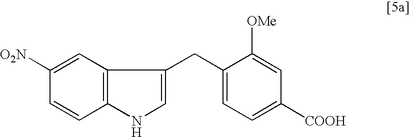
in the preparation of zafirlukast.

21. A process for the preparation of zafirlukast, which comprises:
(a) reacting an alkyl 4-(halomethyl)-3-methoxybenzoate of formula [2a] 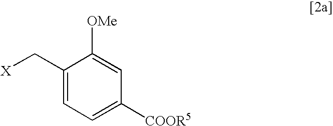
with excess of 5-nitroindole in the presence of zinc halide under conditions promoting alkylation at the 3-position of the indole to yield a mixture comprising an alkyl 3-methoxy-4-(5-nitroindol-3-ylmethyl)benzoate of formula [4a] 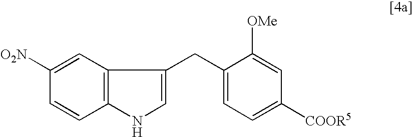
wherein R⁵ is alkyl group and X is halogen; and unreacted 5-nitroindole,
(b) treating the mixture obtained in step (a) with sodium hydroxide to yield a mixture, comprising the monosodium salt of the acid of formula [5a] 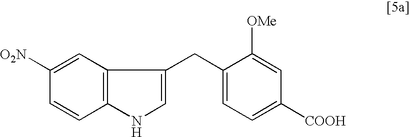
and the unreacted 5-nitroindole,
(c) recovering of the unreacted 5-nitroindole from the mixture, obtained in step (b), and recycling it as starting material to step (a),
(d) isolating the monosodium salt of 3-methoxy-4-(5-nitroindol-3-ylmethyl)benzoic acid in substantially pure solid form and/or acidifying the salt to form 3-methoxy-4-(5-nitroindol-3-ylmethyl)benzoic acid [5a], 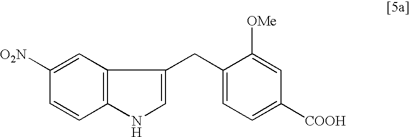
(e) reacting the acid [5a] or it's salt with a methylating agent in the presence of base to form methyl 3-methoxy-4-(1-methyl-5-nitroindol-3-ylmethyl)benzoate of formula [a] 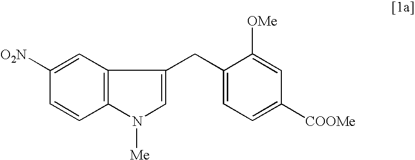
(f) converting the compound [a]obtained in step (e) above into zafirlukast.

22. The monosodium salt of 3-methoxy-4-(5-nitroindol-3-ylmethyl)benzoic acid in substantially pure solid form.
